(19) United States
(12) Patent Application Publication
(10) Pub. No.: US 2009/0311243 A1
(43) Pub. Date: Dec. 17, 2009

(54) Methods for Identifying FZD8 Modulators and the Use of such Modulators for Treating Osteoarthritis

(75) Inventors: Sarah Brockbank, Cheshire (GB); Peter Newham, Cheshire (GB)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 11/915,966

(22) PCT Filed: May 29, 2006
(86) PCT No.: PCT/SE2006/000623

§ 371 Date: Nov. 29, 2007

(30) Foreign Application Priority Data
May 30, 2005 (SE) 0501230-7

(51) Int. Cl. A61K 39/395; C12Q 1/02

(52) U.S. Cl. 424/130.1; 435/29

(57) ABSTRACT

Methods and kits for identifying agents that modulate the function of the Methods and kits for identifying agents that modulate the function of the receptor Fzd8 are provided. Accordingly, the present invention makes available methods for identifying agents useful as modulators of Wnt-Fzd8 signalling. Therapeutic uses of the identified agents for treating osteoarthitis are also provided.

### TECHNICAL FIELD

The present invention relates to the finding that receptor Fzd8 is specifically, differentially and/or highly expressed by osteoarthritic tissue compared to normal tissue. Thus the present invention relates generally to methods of using Fzd8 antagonists in the treatment of chondrocyte and cartilage-related disorders, particularly osteoarthritis (OA). The present invention also relates to the identification of Wnt polypeptides, including Wnt8a, Wnt8b and Wnt3 as ligands for the receptor Fzd8 and the role of Wnt-Fzd8 signalling in regulating cartilage chondrocyte phenotype in osteoarthritis.

### BACKGROUND TO INVENTION

Osteoarthritis (OA) is a degenerative joint disorder which is a leading cause of disability in Western society. It is characterised by progressive abnormalities in the articular cartilage, subchondral bone, synovial fluid, synovial membrane and periarticular structures, in particular muscle. The exact aetiology remains obscure, but abnormalities in the chondrocyte are probably responsible for the onset of disease.

Environmental risk factors for osteoarthritis depend on the joint concerned, but include trauma, nutritional factors (including low dietary intake of vitamins C, D and E), obesity and specific occupational and other activities. It is also clear that genetic factors are important in the aetiology of osteoarthritis.

Currently, osteoarthritis is treated with a combination of pharmacological and non-pharmacological approaches. Pharmacological approaches include the administration of NSAIDs but a number of the drugs used in treatment have associated undesirable side effects.

There therefore remains a need for improved treatment regimes.

Wnt proteins form a family (19 members known to date) of highly conserved secreted signalling molecules that regulate cell differentiation, cell-cell interactions and have key roles in development and disease. Wnt proteins regulate many stages of development, from patterning of the embryo and generation of tissues and cell types, to regulation of cell movements, polarity, axon guidance and synapse formation (Willert, K., Brown, J D., Danenberg, E., Duncan, A W., Weissman, I L., Reya, T., Yates, J R., Nusse, R., (2003) Wnt proteins are lipid modified and can act as stem cell growth factors (Nature, 423, 409-414). At the cellular level they are involved in cell differentiation and proliferation.

In Xenopus soluble Fzd8 CRD has been shown to have patent antagonizing activity of Wnt signalling, as assayed by inhibition of Xenopus secondary axes induced by Xenopus Wnt8 (Deardoff M. et al (1998) Development 125; 2687).

Wnts are hydrophobic lipid-modified proteins that bind to cell-surface receptors of the Frizzled (Fzd) family. Fzds form a family (10 members known to date) of seven transmembrane receptors, which are characterised by an N-terminal cysteine rich domain (CRD) thought to have a role in ligand binding.

In the classical Wnt signalling pathway, Wnts signal through Fzd receptors to β-catenin (canonical pathway) and result in the activation or inhibition of TCF/LEF responsive genes (Nusse, R. (1999), Wnt Targets: Repression and activation, TIG 15(1), 1-3).

In addition, secreted Wnts bind members of the SFRP (secreted Fzd related peptide) family. SFRPs are secreted proteins with homology to the CRD ligand-binding domain of the Fzd receptors and are thought to function as extracellular Wnt inhibitors.

The lack of a 1:1 correlation between individual Wnt and Fzd mutant phenotypes observed in Drosophila may suggest a certain level of redundancy in the Wnt/Fzd system. Work carried out by Nusse in 2002 looking at binding of Wnts to Fzds in drosophila suggested pairings as follows: Wnt1 with Fzd2, Wnt4 with Fzd1 and Fzd2. Wnt8 was tested but did not bind any Fzds in the study with high affinity. (Chi-hwa Wu and Roel Nusse (2002) Ligand Receptor Interactions in the Wnt Signaling Pathway in Drosophila J. Biol. Chem. 277: 41762-41769.

In vitro solid-phase binding assays have demonstrated that soluble Xenopus Wnt8 protein is able to bind to the CRD domain of mouse Fzd8 (CRD-IgG fusions) with an affinity of ˜9 nM (Hsieh, J. C., Rattner, A., Smallwood, P. M. and Nathans, J. (1999). Biochemical characterization of Wnt-frizzled interactions using a soluble, biologically active vertebrate Wnt protein. Proc. Natl. Acad. Sci. USA 96, 3546-3551) and Zebrafish Wnt8b and Zebrafish Fzd8a were shown to functionally interact when expressed exogenously in zebrafish embryos (Kim, S. H. Jimann Shin, Hae-Chul Park, Sang-Yeob Yeo, Sung-Kook Hong, Sangtae Han, Myungchull Rhee, Cheol-Hee Kim, Ajay B. Chitnis and Tae-Lin Huh (2002). Specification of an anterior neuroectoderm patterning by Frizzled8a-mediated Wnt8b signalling during late gastrulation in zebrafish. Development, 129(19): 4443-4455).

However, to date, a clear association between human Fzd8 and a particular Wnt ligand has not been shown.

### SUMMARY OF THE INVENTION

The inventors have now determined that Fzd8 plays an important role in the development and progression of osteoarthritis. They have also determined that Fzd8 is a receptor for Wnt8a.

Primary articular cartilage chondrocytes stimulated with Wnt8a respond by modulating the expression of genes associated with a change in phenotype from a resting state to a more hypertrophic state, a change normally associated with the process of endochondral ossification.

Endochondral ossification is the formation of calcified bone following remodelling of a cartilage scaffold model and is the normal process of long bone growth during development. Cartilage chondroyte phenotype changes occur during this process and lead to the accumulation of mature hypertrophic chondrocytes that express and secrete matrix proteins such as collagen X, resulting in a mineralised extracellular matrix and ultimately the formation of new bone. In normal, fully developed articular cartilage, where further endochondral ossification is undesirable, chondrocyte differentiation is negatively regulated. However, in osteoarthritis, this regulation is apparently lost and deep zone calcified cartilage, found at the interface between non-mineralised articular cartilage and bone, expands and extends into the deep zone cartilage, compromising the mechanical properties of the overlying cartilage matrix. Thus the Wnt/Fzd8 pathway plays a role in stimulating articular cartilage chondrocyte differentiation and hypertrophy leading to a more calcified deep zone extracellular matrix that ultimately contributes to the loss of cartilage integrity, cartilage erosion and the joint space narrowing observed during osteoarthritis. Thus the present invention relates to targetting this pathway with agents that modulate its activity so as to treat osteoarthritis. The present invention also relates to the use of antagonists of the Wnt\Fzd8 pathway for the treatment of osteoarthritis. It is believed that by reducing or inhibiting chondrocyte differentiation by targetting this pathway the formation of mineralised cartilage will be reduced and the thus the progression of osteoarthritis slowed or halted.

Accordingly, in one aspect, the invention provides a method for inhibiting the Fzd8 pathway in a chondrocyte cell, wherein the method comprises contacting the chondrocyte cell with a Wnt/Fzd8 pathway antagonist, which causes reduction or inhibition of chondrocyte differentiation and/or hypertropy. In one embodiment, the antagonist is a Fzd8 antagonist that binds to Fzd8 on a chondrocyte cell. Preferably the Fzd8 antagonist is an antibody that binds to Fzd8. In another embodiment, an antagonist of the Fzd8 ligand can be used, for example, an antibody that binds to a Wnt ligand of Fzd8, for example, Wnt8a. In another aspect the antagonist is the CRD domain of Fzd8 which binds to the Wnt ligand of Fzd8, thereby preventing or reducing the level of Wnt ligand binding to Fzd8.

In another aspect, the invention provides a method of therapeutically treating a mammal having osteoarthritis, wherein the method comprises administering to the mammal a therapeutically effective amount of a Fzd8 antagonist, thereby resulting in the effective therapeutic treatment of osteoarthritis. Preferably the Fzd8 antagonist is an antibody, more preferably an antibody that binds to the CRD domain of Fzd8.

The invention also relates to the identification of Wnt polypeptides, including Wnt8a, Wnt8b and Wnt3 as natural ligands of the human Fzd8 receptor. The invention encompasses the use of the interaction between Wnt polypeptides, including Wnt8a, Wnt8b and Wnt3 polypeptides and Fzd8 polypeptides as the basis for screening assays for agents that modulate the activity of the Fzd8 receptor. Furthermore, the Wnt/Fzd8 interaction, in particular the Wnt8a interaction with Fzd8 is demonstrated to be associated with the development of osteoarthritis. Accordingly, the invention also encompasses diagnostic assays based upon the Wnt/Fzd8 interaction, as well as kits for performing diagnostic and screening assays.

Accordingly, in one aspect, the invention provides a method of identifying an agent that modulates the function of Fzd8, said method comprising:
a) contacting a Fzd8 polypeptide with a Wnt polypeptide in the presence or absence of a candidate modulator under conditions permitting the binding of said Wnt polypeptide to said Fzd8 polypeptide; and
b) measuring the binding of said Fzd8 polypeptide to said Wnt polypeptide wherein a decrease in binding in the presence of said candidate modulator, relative to the binding in the absence of said candidate modulator, identifies said candidate modulator as an agent that modulates the function of Fzd8.

As used herein, the term “Fzd8 polypeptide” refers to a polypeptide having an amino acid sequence as set out in EMBL Acc No AX367099 or to a polypeptide having at least 70%, and preferably 80%, 90%, 95% or higher, amino acid identity thereto, as well as to a polypeptide having Fzd8 function or activity such as the polypeptide binding to a Wnt polypeptide, such as Wnt8a, Wnt8b and Wnt3 polypeptide or a functional fragment thereof. “Fzd8 polypeptide” also refers to shortened forms, mutants, derivatives and active fragments of polypeptide wherein said forms and fragments retain Fzd8 activity. In one embodiment, said fragments include the Wnt binding site of Fzd8.

By “the function of Fzd8” or “Fzd8 activity” means Fzd8 binding activity and, in particular, the ability to bind to the ligand Wnt such as Wnt8a, Wnt8b and Wnt3. Binding activity between two proteins can be detected by a number of techniques which will be familiar to those skilled in the art. “the function of Fzd8” also refers to Fzd8 signalling activity.

As used herein, the term “Wnt polypeptide” refers to a polypeptide having an amino acid characteristic of a Wnt family protein. Thus, for example, “Wnt polypeptide” includes Wnt8a, Wnt8b and Wnt3 polypeptides. “Wnt8a polypeptide”, for example, refers to a polypeptide having an amino acid sequence as set out in EMBL Acc No AB057725 or to a polypeptide having at least 70%, and preferably 80%, 90%, 95% or higher, amino acid identity thereto, as well as to a polypeptide having Wnt8a activity such as the polypeptide binding to Fzd8 polypeptide or a functional fragment thereof. “Wnt polypeptide” also refers to shortened forms and active fragments of polypeptide wherein said forms and fragments retain Wnt activity.

By “Wnt activity” or function means Wnt ligand activity and, in particular, its ability to bind to Fzd8.

Methods for detecting binding include surface plasmon resonance (SPR), for example using sensors available from Biacore AB, fluorescence resonance energy transfer (FRET), fluorescence polarization measurement, electrochem luminescence and chemiluminescence, a biosensor assay and so forth.

Binding assays for Wnts and Fzds are detailed in this reference, where Wnt8 is able to bind to the CRD domain of Fzd8 with an affinity of 9 nM (Hsieh, J. C., Rattner, A., Smallwood, P. M. and Nathans, J. (1999) Biochemical characterization of Wnt-frizzled interactions using a soluble, biologically active vertebrate Wnt protein. Proc. Natl. Acad. Sci. USA 96, 3546-3551).

In another aspect of the invention there is provided a method of identifying an agent that modulates the function of Fzd8, said method comprising:
a) contacting a Fzd8 polypeptide with a Wnt polypeptide in the presence or absence of a candidate modulator under conditions permitting the binding of said Wnt polypeptide to said Fzd8 polypeptide; and
b) measuring the signalling activity of said Fzd8 polypeptide to said Wnt polypeptide, wherein a change in activity in the presence of said candidate modulator, relative to the activity in the absence of said candidate modulator, identifies said candidate modulator as an agent that modulates the function of Fzd8.

As described herein, one suitable method for measuring Fzd8 signalling activity is to measure the accumulation, nuclear translocation or phosphorylation status of β-catenin. Measurements can be made using techniques such as immunofluorescence although other suitable methods will be familiar to those skilled in the art. Other suitable methods for measuring Fzd8 signalling activity include measuring downstream signalling events including gene expression mediated through β-catenin. As described herein, β-catenin modulates gene expression through the TCF/LEF family of transcriptional activators/repressors. Accordingly, activity may also be measured by detecting subsequent TCF/LEF transcriptional activity. Genes whose expression is modulated include Col α1(II), Col α1(IX), Col α1(XI), SOX9, aggrecan, Col α1(X), fibulin, bone matrix GLA protein, chondromodulin and VEGF.

Another suitable assay involves a reporter gene assay in which a Fzd8 responsive element is joined to a reporter construct. Suitable reporter gene constructs will be familiar to those skilled in the art.

In one embodiment, an agent that modulates function is detected if said binding or said signalling activity is at least 50% of the amount induced by Wnt at its EC₅₀ in the absence of said candidate agent.

Similarly, in an alternative aspect of the invention, there is provided a method of identifying an agent that modulates the function or activity of Wnt, said method comprising:
a) contacting a Fzd8 polypeptide with a Wnt polypeptide in the presence or absence of a candidate modulator under conditions permitting the binding of said Wnt polypeptide to said Fzd8 polypeptide; and
b) measuring the binding of said Fzd8 polypeptide to said Wnt polypeptide, wherein a decrease in binding in the presence of said candidate modulator, relative to the binding in the absence of said candidate modulator, identifies said candidate modulator as an agent that modulates the function or activity of Wnt.

Preferably the Wnt polypeptide for use in the method is selected from Wnt3, Wnt 8a or Wnt8b.

In a preferred embodiment of any aspect of the invention, the method or assay is a membrane assay.

Suitably, a method in accordance with this aspect of the invention is a cellular assay such as a whole cell assay. Such a cellular assay preferably involves the steps of expressing Fzd8 in a cell line in the presence of Wnt8a, contacting said cell with candidate modulator and measuring β-catenin accumulation nuclear translocation or phosphorylation status. In one embodiment, said whole cell assay can comprise co-transfecting a cell or cell line with Fzd8 and Wnt8a.

Suitably, a method in accordance with the invention may be set up as a screening assay. Screening can be, for example in vitro, in cell culture, and/or in vivo. Biological screening assays preferably centre on activity-based response models, binding assays (which measure how well a compound binds), and bacterial, yeast and animal cell lines (which measure the biological effect of a compound in a cell). Suitable assays are described herein. The assays can be automated for high capacity-high throughput screening (HTS) in which large numbers of modulators can be tested to identify compounds or modulators with the desired activity.

Current screening technologies are described in Handbook of Drug Screening, edited by Ramakrishna Seethala, Prabhavathi B. Fernandes. New York, N.Y., Marcel Dekker, (2001).

In one embodiment of any aspect of the invention, the Fzd8 or Wnt polypeptide may be detectably labelled i.e. the polypeptide has a structural modification that incorporates a functional group (label) that can be readily detected. Detectable labels include fluorescent compounds, isotopic compounds, biotin, enzymes, proteins for which antisera or monoclonal antibodies are available, among others.

As used herein, the term “candidate modulator” includes, but is not limited to, a compound which may be obtainable from or produced by any suitable source, whether natural or not.

In another aspect of the invention there is provided a method for identifying an agent that modulates the function or activity of a Wnt polypeptide using a method in accordance with any of the above aspects.

The candidate modulator or compound may be designed or obtained from a library of compounds, which may comprise peptides, as well as other compounds, such as small organic molecules and particularly new lead compounds. By way of example, the candidate modulator compound may be a natural substance, a biological macromolecule, or an extract made from biological materials—such as bacteria, fungi, or animal (particularly mammalian) cells or tissues, an organic or an inorganic molecule, a synthetic candidate modulator, a semi-synthetic candidate compound, a structural or functional mimetic, a peptide, a peptidomimetic, a derivatised candidate compound, a peptide cleaved from a whole protein, or a peptide synthesised synthetically, for example, either using a peptide synthesiser or by recombinant techniques or combinations thereof, a recombinant candidate modulator, a natural or a non-natural candidate compound, a fusion protein or equivalent thereof and mutants, derivatives or combinations thereof. Other candidate modulator compounds can include antibodies, aptamers and nucleic acid molecules including siRNA or antisense compounds. The candidate compound may even be a compound that is a modulator of Fzd8 or Wnt, such as a known inhibitor of Fzd8 or a Wnt polypeptide, that has been modified in some way eg. by recombinant DNA techniques or chemical synthesis techniques.

Typically, the candidate compound will be prepared by recombinant DNA techniques and/or chemical synthesis techniques.

Once a candidate compound capable of interaction with Fzd8 or a Wnt polypeptide, particularly the Wnt 8a or Wnt 8b polypeptide, has been identified, further steps may be carried out to select and/or to modify the candidate compounds and/or to modify existing compounds, such that they are able to modulate Fzd8 or a Wnt polypeptide.

In one aspect, the modulator of Fzd8 or a Wnt polypeptide may act as a model (for example, a template) for the development of other compounds.

A further aspect relates to the use of candidate compounds or Fzd8 or a Wnt polypeptide modulators identified by the assays and methods of the invention in one or more model systems, for example, in a biological model, a disease model, or a model for Fzd8 inhibition. Such models may be used for research purposes and for elucidating further details of the biological, physicochemical, pharmacological and/or pharmacokinetic activity of a particular candidate compound. By way of example, the candidate compounds or Fzd8 or Wnt polypeptide modulators of the present invention may be used in biological models or systems in which Fzd8 signalling is known to be of particular significance.

In another aspect there is provided an agent identified using a method in accordance with the invention.

Another aspect of the invention relates to a process comprising the steps of:
(a) performing the method according to the invention, or an assay according to the invention;
(b) identifying one or more modulators of Fzd8 or a Wnt polypeptide; and
(c) preparing a quantity of said one or more Fzd8 or Wnt polypeptide modulators.
   Preferably the Wnt polypeptide modulator is a modulator of Wnt8a or Wnt8b.

A further aspect of the invention relates to a process comprising the steps of:
(a) performing the method according to the invention, or an assay according to the invention;
(b) identifying one or more Fzd8 or Wnt polypeptide modulators; and
(c) preparing a pharmaceutical composition comprising said one or more identified Fzd8 or Wnt polypeptide modulators.
   Preferably the Wnt polypeptide modulator is a modulator of Wnt8a or Wnt8b.

A further aspect relates to a process comprising the steps of:
(a) performing the method according to the invention, or an assay according to the invention;
(b) identifying one or more Fzd8 or Wnt polypeptide modulators;
(c) modifying said one or more Fzd8 or Wnt polypeptide modulators; and
(d) optionally preparing a pharmaceutical composition comprising said one or more Fzd8 or Wnt polypeptide modulators.
   Preferably the Wnt polypeptide modulator is a modulator of Wnt8a or Wnt8b.

In another aspect of the invention there is provided a modulator of Fzd8 or a Wnt polypeptide for use as a medicament.

Preferably the modulator use as a medicament is a modulator of Wnt8a or Wnt8b.

Suitable modulators or candidate compounds include modulators that can down regulate Fzd8 or Wnt gene expression, particularly Wnt8a or Wnt8b. Down regulation of gene expression can be achieved through the administration of compounds such as siRNA molecules. Suitable siRNA molecules for the down regulation of expression of Fzd8 are described herein.

Other modulators include antibodies, aptamers, antisense or siRNA molecules and so forth.

In a yet further aspect of the invention there is provided use of a modulator of Fzd8 or Wnt polypeptide, particularly Wnt8a or Wnt 8b polypeptide, in the preparation of a medicament for use in the treatment of OA.

Suitably said modulator is a compound identified in any method in accordance with the invention.

As identified herein, overexpression of human sFRP3 (FrzB) polypeptide can block Fzd8 signalling activity in response to a Wnt polypeptide. Accordingly, in another aspect of the invention there is provided a composition comprising sFRP3 (FrzB) polypeptide, or functional fragment or derivative thereof, for use as a medicament.

In another aspect of the invention there is provided a composition comprising the cysteine rich domain of human Fzd8, or a functional fragment or derivative thereof, in the preparation of a medicament for use in the treatment of OA. The cysteine rich domain is the N-terminal conserved domain of 120 amino acids of human Fzd8, which contains 10 invariant cysteines, (EMBLAccNo AB043703)

In another aspect of the invention there is provided a method of diagnosing a disease characterised by dysregulation of Fzd8 signalling comprising the steps of detecting Fzd8 expression in a sample and comparing said expression with expression in a normal tissue.

As described herein, Fzd8 expression is upregulated in osteoarthritic cartilage. Accordingly, in another aspect of the invention there is provided a method of diagnosing OA through detecting Fzd8 expression in a sample tissue compared with a normal tissue.

Suitably detection of Fzd8 expression can be through techniques such as PCR, in situ hybridisation or immunochemistry although other techniques will be familiar to those skilled in the art.

In another aspect there is provided a kit for screening for agents that modify Fzd8 activity.

In a yet further aspect there is provided a kit for diagnosis.

### BRIEF DESCRIPTION OF THE TABLES AND FIGURES

Table 1. Primers and probes for Fzd8, Wnt8a, COL9A1, COL11A1, COLX, BGLAP, VEGF and Fzd7 (Assay on Demand, ABI) and GAPDH (pre-developed primer/probe mix) were obtained as a pre-formulated 20× mix from Applied Biosystems.

Table 2. Effects of Wnt8a overexpression in osteoarthritic chondrocytes. Chondrocytes were infected with adenovirus (Adv): Adv-Wnt8a or Adv-RFP (control) and Wnt8a specific gene expression changes were monitored using quantitative RT-PCR at intervals for up to 42 days. Wnt8a regulated gene changes are presented as fold induced (upregulated genes) or fold-reduced (down-regulated genes) relative to gene expression level of control (Adv-RFP) infected cells.

### FIGURE LEGENDS

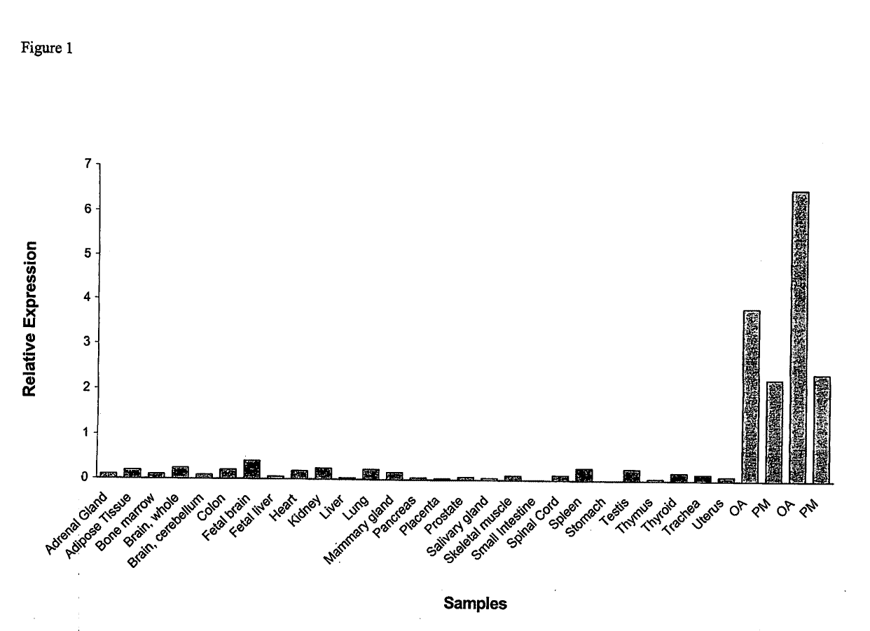

FIG. 1. Human Fzd8 Quantitative RT-PCR in normal human tissues and human articular cartilage. Fzd8 expression was normalised to 18S RNA.

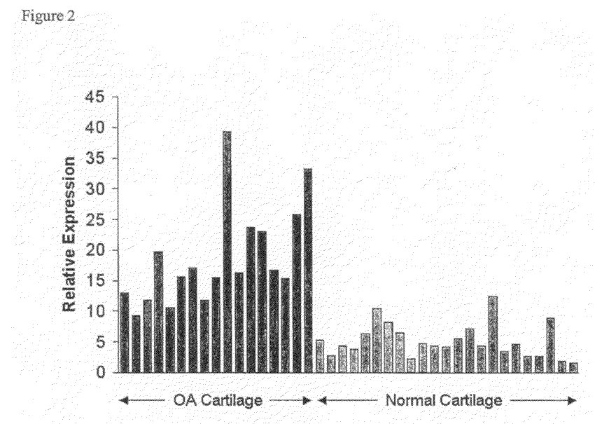

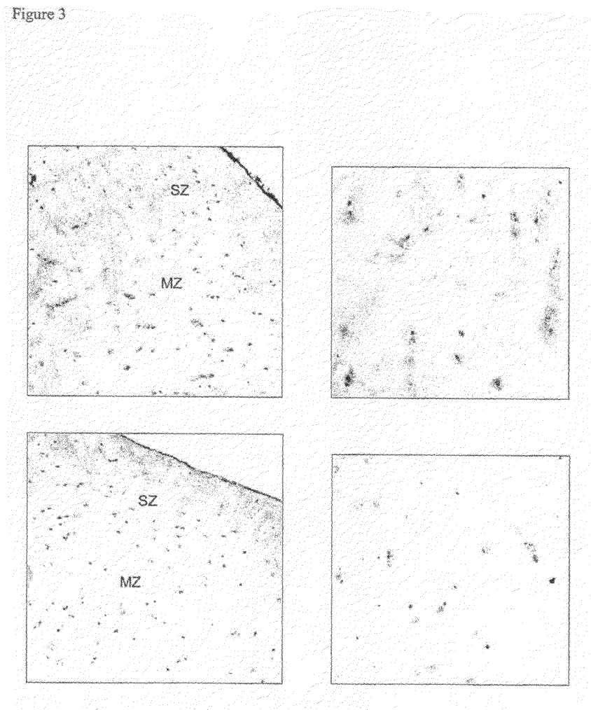

FIG. 2. Human Fzd8 Quantitative RT-PCR in normal (post-mortem) and osteoarthritic articular cartilage. Samples include RNA derived from medial and lateral femoral condyles and tibal plateaus. Fzd8 expression was normalised to 18S RNA.

FIG. 3. Fzd8 Immunohistochemistry. Human Fzd 8 was detected in human OA articular cartilage using rabbit anti-human Fzd8 polyclonal antibody in the absence (top) or presence (bottom) of Fzd8 immunisation peptide. In certain examples Mid/Deep zone chondrocytes demonstrated strong, specific Fzd8 reactivity (DAB chromogen). Images are 10× magnification (left) or 20× magnification of mid-zone images (right). SZ=superficial zone. MZ=mid zone.

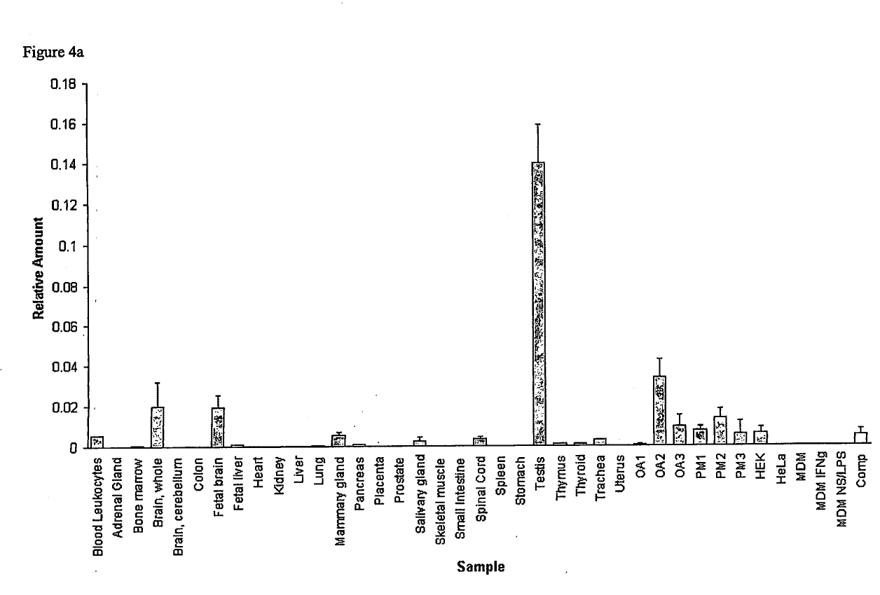

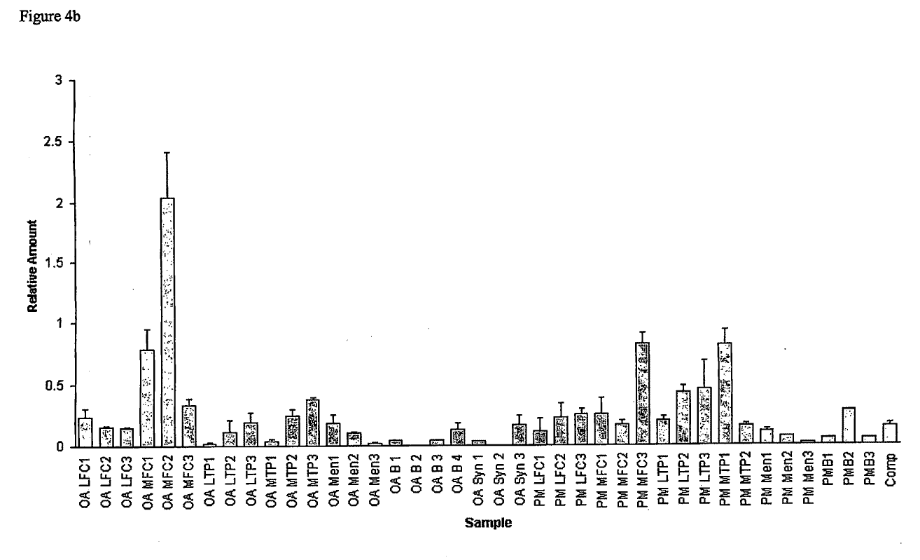

FIG. 4. Human Wnt8a quantitative RT-PCR in normal human tissues (top) and human articular cartilage/bone sub-compartments (bottom). Wnt expression was normalised to 18s RNA. OA prefix=osteoarthritis articular cartilage, PM pre-fix=post-mortem articular cartilage (control). Top panel suffixes: LFC=lateral femoral condyle; MFC=medial femoral condyle, LTP=lateral tibial plateau, MTP=medial tibial plateau, B=sub-chondral bone, syn=synovium. Comp=control RNA from diverse human tissue set. Bottom panel suffixes: #=articular cartilage sample number, MDM=monocyte-derived macrophage, MDMIFNg=IFN-γ stimulated monocyte-derived macrophage, MDM NS/LPS=LPS stimulated monocyte-derived macrophage. Comp=control RNA from diverse human tissue set.

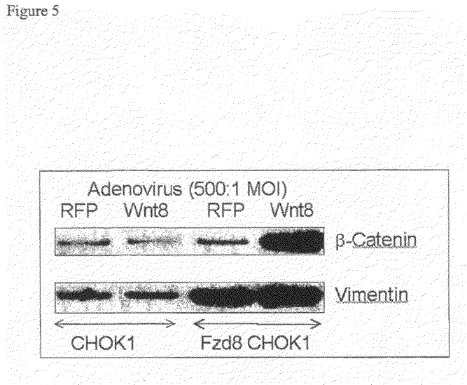

FIG. 5. CHOK1 and Fzd8-CHOK1 (stably expressing Fzd8) β-catenin western blot of following infection by either control (RFP) or Wnt8a adenovirus at indicated MOI. Vimentin western blotting was used to control for protein loading.

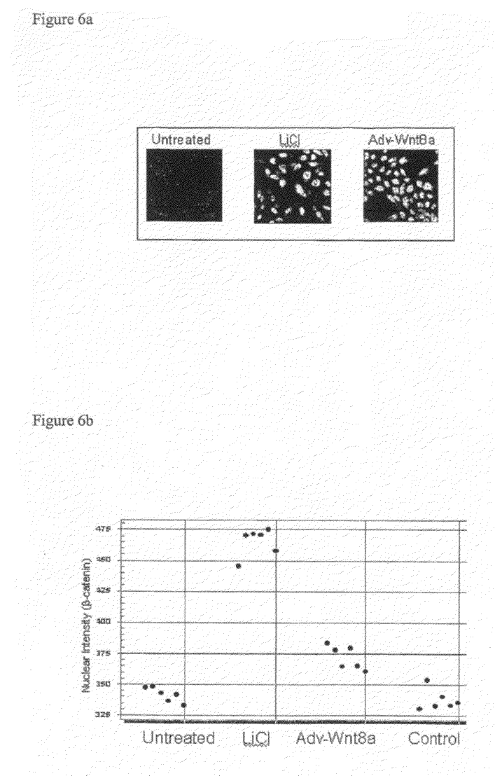

FIG. 6. β-catenin nuclear translocation. a) Immunofluorosecence imaging of CHOK1-Fzd8 β-catenin. Cells were stimulated with LiCl (30 mM), Adv-Wnt8a (MOI 1000:1) for 24 h. b) CHOK1-Fzd8 β-catenin nuclear staining quantitated using Arrayscan (Cellomics) nuclear localisation software. Six replicate CHOK1-Fzd8 wells were untreated or stimulated with LiCl (30 mM), infected with Adv-Wnt8a (MOI 1000:1) or infected with control Adv-RFP (MOI 1000:1) for 24 h.

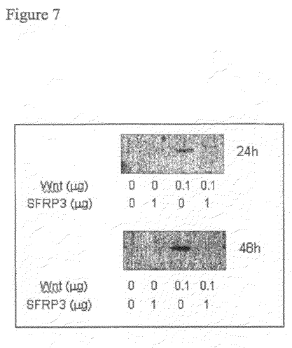

FIG. 7. Fzd8:CHOK1 cells were transfected with indicated amounts of pcDNA3 encoding either Wnt8a or SFRP3 alone or costransfected with pcDNA3-Wnt8a and pcDNA3-SFRP3. β-catenin accumulation (equivalent protein loading in each lane) was detected after 24 h or 48 h as indicated.

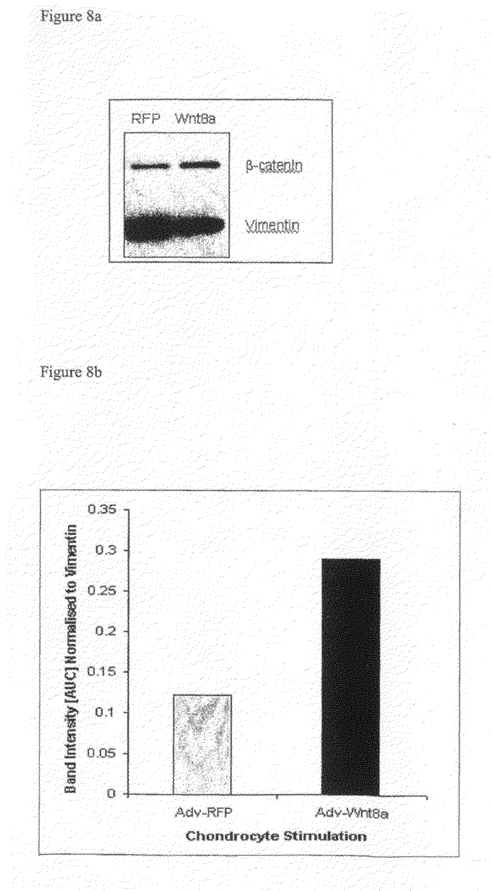

FIG. 8. Primary OA articular cartilage chondrocyte β-catenin western blot following infection by either control (RFP) or Wnt8a adenovirus for 72 h. Vimentin western blotting was used to control for protein loading and normalise data. a) β-catenin and vimentin detection following SDS-PAGE and ECL western blot; b) quantitation of β-catenin protein level following normalisation to vimentin.

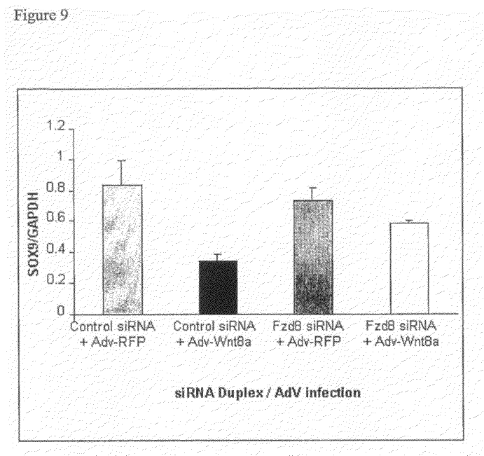

FIG. 9. Wnt8a-induced chondrocyte gene expression changes are mediated by Fzd8. Primary chondrocytes were transfected with 25 nM-100 nM siRNA using Atufect lipid (Atugen) and 48 h post-transfection were infected with Adv-Wnt8a (MOI=500:1). Effects on gene expression were monitored by quantitative PCR 72 h post-transfection. Sample data: Wnt8a-suppressed SOX9 expression. SOX9 mRNA levels 72 h post-transfection were normalised versus GAPDH mRNA. Wnt8a reduced SOX9 mRNA, and this was rescued (52% effect) when Fzd8 was knocked down by Fzd8 siRNA. Control siRNA=irrelevant siRNA duplex. Fzd8 siRNA=Fzd8-specific siRNA duplex. Data presented with siRNA duplexes used at 25 nM.

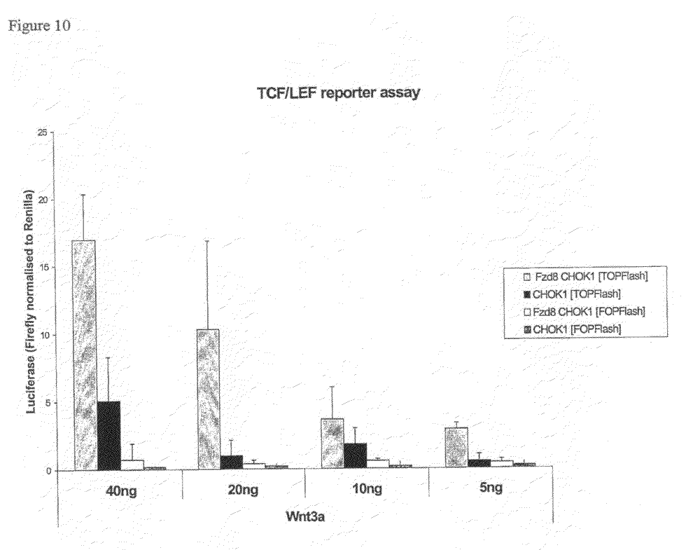

FIG. 10

CHOK1 cells and Fzd8:CHOK1 cells expressing TOPFlash or FOPFlash were treated with 5-40 ng Wnt3a. TCF/LEF reporter gene activity was measured as Firefly luciferase activity normalised to Renilla luciferase activity.

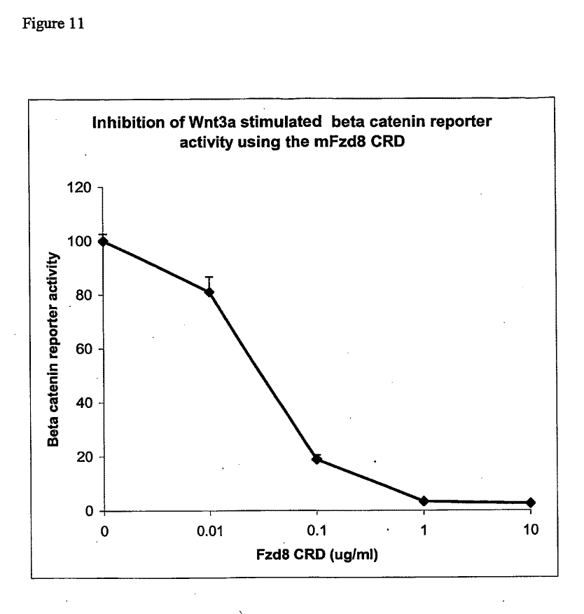

FIG. 11

Fzd8 CHOK1 cells expressing TOPFlash were treated with Wnt3a preadsorbed with 0-10 ug/ml Fzd8 CRD Fc fusion protein. TCF/LEF reporter gene activity was measured as Firefly luciferase activity.

### DETAILED DESCRIPTION

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods. See, generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999)4^{th} Ed, John Wiley & Sons, Inc.; as well as Guthrie et al., Guide to Yeast Genetics and Molecular Biology, Methods in Enzymology, Vol. 194, Academic Press, Inc., (1991), PCR Protocols: A Guide to Methods and Applications (Innis, et al. 1990. Academic Press, San Diego, Calif.), McPherson et al., PCR Volume 1, Oxford University Press, (1991), Culture of Animal Cells: A Manual of Basic Technique, 2nd Ed. (R. I. Freshney. 1987. Liss, Inc. New York, N.Y.), and Gene Transfer and Expression Protocols, pp. 109-128, ed. E. J. Murray, The Humana Press Inc., Clifton, N. J.). These documents are incorporated herein by reference.

Assays for the Identification of Agents that Modulate Binding of Fzd8 to a Wnt Polypeptide.

Agents that modulate the activity of Fzd8 can be identified in a number of ways that take advantage of the interaction of the receptor with a Wnt polypeptide. For example, the ability to reconstitute Fzd8/Wnt polypeptide binding either in vitro as fractionated cellular components, on cultured cells or in vivo provides a target for the identification of agents that disrupt that binding. Assays based on disruption of binding can identify candidate modulators from a range of sources as described herein.

Modulators of Fzd8/Wnt polypeptide binding can then be screened using a binding assay or a functional assay that measures downstream signaling through the receptor. Both binding assays and functional assays are validated using a Wnt polypeptide.

Another approach that uses the Fzd8/Wnt polypeptide interaction more directly to identify agents that modulate Fzd8 function measures changes in Fzd8 downstream signaling induced by candidate agents or candidate modulators. These functional assays can be is performed in isolated cell membrane fractions or on cells expressing the receptor on their surfaces.

One skilled in the art can readily amplify a polypeptide sequence from a sample containing mRNA encoding that protein through basic PCR and molecular cloning techniques using primers or probes designed from the known sequences.

The expression of recombinant polypeptides is well known in the art. Those skilled in the art can readily select vectors and expression control sequences for the expression of Fzd8/Wnt polypeptide polypeptides useful according to the invention in eukaryotic or prokaryotic cells.

Fzd8 must be associated with cell membrane or detergents like synthetic liposomes in order to have binding or signaling function. Methods for the preparation of cellular membrane fractions are well known in the art, e.g. the method reported by Hubbard & Cohn, 1975, J. Cell Biol. 64: 461-479, which is incorporated herein by reference. In order to produce-membranes-comprising Fzd8, one need only apply such techniques to cells endogenously or recombinantly expressing Fzd8. Alternatively, membrane-free Fzd8 can be integrated into membrane preparations by dilution of detergent solution of the polypeptide (see, e.g., Salamon et al., 1996, Biophys. J. 71: 283-294, which is incorporated herein by reference).

Frizzled-8 CRD can be produced as a soluble protein by cloning the cysteine rich domain into any one of a number of well known eukaryotic expression vectors and expressing the protein in any one of a number of well known eukaryotic cell lines similarly to the methods described by Dann et al Nature 412 (2001) 86-90. The soluble cysteine rich domain can be produced by cloning the gene encoding a protein from amino acid 1 to approximately 151-173. A C-terminal fusion well known to those skilled in the art such as 6 histidines or an immunoglobulin Fc domain can be added in order to facilitate the purification, for example, prior to using the CRD in order to raise blocking (antagonist) antibodies.

As with Fzd8, Wnt polynucleotides can be cloned through standard PCR and molecular cloning techniques using the known sequences as a source of amplification primers or probes. Similarly, cloned Wnt polypeptides can be expressed in eukaryotic or prokaryotic cells as known in the art. As a non-limiting example, an adenoviral vector for expressing Wnt8a is described herein.

Further, if desired for a given assay or technique, Wnt polypeptides useful according to the invention can be produced as fusion proteins or tagged proteins. For example, either full length Wnt polypeptide or a portion thereof (i.e., at least 10 amino acids, preferably at least 20 amino acids or more, up to one amino acid less than full length Wnt polypeptide) can be fused to Glutathione-S-Transferase (GST), secreted alkaline phosphatase (SEAP), a FLAG tag, a Myc tag, or a 6X-His peptide to facilitate the purification or detection of the Wnt polypeptide. Methods and vectors for the production of tagged or fusion proteins are well known in the art, as are methods of isolating and detecting such fused or tagged proteins.

Wnt polypeptides and particularly truncated forms can also be prepared by chemical synthesis as known in the art.

Recombinant Wnt polypeptides can be used in purified form. Alternatively, conditioned medium from Wnt transfected cells can be used. The amounts of Wnt necessary in a given binding or functional assay according to the invention will vary depending upon the assay, but will generally use 1 pM to 1 nM of labeled and 10 pM to 1 uM of unlabeled Wnt per assay.

Wnt polypeptide can be labelled to facilitate detection of binding. Labelling can be through incorporation of radiolabeled amino acids in the medium during synthesis, fluorescent labels and so forth.

Ligand binding assays include assays in which cells expressing Fzd8, membrane extracts from such cells, or immobilized lipid membranes comprising Fzd8 are exposed to a labeled Wnt polypeptide and candidate compound. Following incubation, the reaction mixture is measured for specific binding of the labeled Wnt polypeptide to the Fzd8 receptor.

Binding assays can include displacement assays where displacement of Wnt is measured in the presence or absence of the candidate modulator compound. Other methods for detecting binding include surface plasmon resonance (SPR), for example using sensors available from Biacore AB, fluorescence resonance energy transfer (FRET), fluorescence polarization measurement, electrochem luminescence and chemiluminescence, a biosensor assay and so forth.

Assays for the Identification of Agents that Modulate Fzd8 Signalling Activity

Assays for screening for compounds that modulate Fzd8 activity can involve cells expressing Fzd8 being incubated in the presence or absence of a candidate modulator compound in the presence of a Wnt polypeptide, and a signalling activity of Fzd8 is measured.

Signalling by Fzd8 can be measured by measuring an accumulation, nuclear translocation or phosphorylation status of β-catenin using methods such as those described herein. Fzd8 signalling activity can also be measured by measuring TCF/LEF reporter activity as described herein and gene expression downstream. Methods for measuring gene expression include techniques for detecting mRNA expression such as PCR or microarray techniques. Gene expression may also be measured by detecting protein expression.

### Cellular Assays

Cellular assays may be performed by generating a cell or cell line that comprises nucleotide sequences that are of use in the present invention, for example, nucleotide sequences encoding Fzd8 or a Wnt polypeptide.

Such cells may be transformed or transfected with a nucleotide sequence contained in a vector e.g. a cloning vector. Preferably, said nucleotide sequence is carried in a vector for the replication and/or expression of the nucleotide sequence. The cells will be chosen to be compatible with the said vector and may for example be prokaryotic (for example bacterial), fungal, yeast or plant cells.

Transfection can also be achieved through the introduction of a viral vector comprising the sequence of interest. For example, an adenoviral vector, as described herein.

In a preferred embodiment, the host cells are mammalian cells, such as CHO-K1 cells, HEK293 cells or chondrocytes.

### Animal Model

Osteoarthritis (OA) is a chronic joint disease characterized by cartilage destruction, subchondral bone sclerosis, and osteophytosis. The rat menisectomy would be an appropriate disease model to measure a hypertrophy biological effect, in this case an osteophyte formation endpoint where both the size and degree of calcification would be measured.

Compounds or antibodies which are potential candidate drugs would then be tested in the Dunkin Hartley guinea pig spontaneous OA model to confirm that inhibitors have a chondroprotective effect on OA pathogenesis. Characteristic features integral to the disease process include subchondral remodeling and Zone of Calcified Cartilage (ZCC) duplications, such changes can be measured in this model.

A positive result would be a reduction of osteophytosis, a reduction in cartilage degradation. However, the magnitude or absolute measure of the effect is not important, but the reduction in the effect needs to be statistically significant between control (vehicle) and treated (p value of <0.05).

### Modulators

As herein, the term “modulating” or “modulates” refers to preventing, suppressing, inhibiting, alleviating, restorating, elevating, increasing or otherwise affecting Fzd8 or Wnt polypeptide activity. Suitably Fzd8 activity is Fzd8 signalling activity.

The term “modulator” may refer to a single entity or a combination of entities.

The modulator may be an antagonist or an agonist of said Fzd8 or said Wnt polypeptide.

As used herein, the term “agonist” means any entity, which is capable of interacting (eg. binding) with Fzd8 or Wnt resulting in an increased or modified biological response. Suitably an agonist can be a protein ligand, peptide, chemokine, chemoattractant, lipid derivative or cytokine. For example, Wnt8a, one of the natural ligands for Fzd8, is an agonist of Fzd8.

As used herein, the term “antagonist” means any entity, which is capable of interacting (eg. binding) with Fzd8 or Wnt resulting in a decreased biological response to the agonist. For example, by reducing agonist binding to the receptor.

Preferably, the modulators of the present invention are antagonists of Fzd8 and modulate Fzd8 to reduce ligand binding and activation of Fzd8. Preferably the antagonist is an antibody that binds to Fzd8, also referred to herein as “antagonist antibody of Fzd8” or “Fzd8 antagonist antibody”. In another embodiment, the Fzd8 modulators are activators, or agonists of Fzd8, that modulate Fzd8 to increase activation of Fzd8.

The modulator may be an organic compound or other chemical. The modulator may be a compound, which is obtainable from or produced by any suitable source, whether natural or artificial. The modulator may be an amino acid molecule, a polypeptide, or a chemical derivative thereof, or a combination thereof. The modulator may even be a polynucleotide molecule, which may be a sense or an anti-sense molecule or an siRNA molecule.

The modulator may be designed or obtained from a library of compounds, which may comprise peptides, as well as other compounds, such as small organic molecules.

By way of example, the modulator may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetic, a derivatised agent, a peptide cleaved from a whole protein, or a peptide synthesised synthetically (such as, by way of example, either using a peptide synthesiser or by recombinant techniques or combinations thereof, a recombinant agent, an antibody, a natural or a non-natural agent, a fusion protein or equivalent thereof and mutants, derivatives or combinations thereof).

Typically, the modulator will be an organic compound. Typically, the organic compounds will comprise two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. For some applications, preferably the modulator comprises at least one cyclic group. The cyclic group may be a polycyclic group, such as a non-fused polycyclic group. For some applications, the modulator comprises at least the one of said cyclic groups linked to another hydrocarbyl group.

The modulator may contain halo groups, for example, fluoro, chloro, bromo or iodo groups, or one or more of alkyl, alkoxy, alkenyl, alkylene and alkenylene groups, each of which may be branched or unbranched.

Preferably, the modulator of the present invention may be prepared by chemical synthesis techniques which are familiar to those skilled in the art.

The modulator may be used in combination with one or more other pharmaceutically active agents. If combinations of active agents are administered, then they may be administered simultaneously, separately or sequentially.

In one embodiment the modulator is the cysteine rich domain (CRD) of human Fzd8, or a functional fragment or derivative thereof. The cysteine rich domain comprises the N-terminal conserved domain of 120 amino acids of human Fzd8, which contains 10 invariant cysteines, see EMBLAccNo AB043703.

In one embodiment the modulator is an antibody, preferably an antibody that binds to Fzd8 or a Wnt ligand of Fzd8, e.g. Wnt8a. In a preferred embodiment the antibody is a Fzd8 antagonist antibody.

### Antibodies

The term “antibody” is used in the broadest sense and specifically covers, for example, single monoclonal antibodies (including antagonist, binding and/or neutralizing antibodies), antibody compositions with polyepitopic specificity, polyclonal antibodies, single chain anti-FZD8 polypeptide antibodies, and fragments of antibodies (see below) as long as they exhibit the desired biological or immunological activity.

An antibody useful in methods of the invention is one which has been identified and separated and/or recovered from a component of its natural environment. Contaminant components of its natural environment are materials which would interfere with therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by gel electrophoresis such as SDS-PAGE under reducing or nonreducing conditions using, for example, Coomassie blue or silver stain. The basic 4-chain antibody unit is a heterotetrameric glycoprotein composed of two identical light (L) chains and two identical heavy (H) chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated α, δ, ε, γ, and μ, respectively. The γ and a classes are further divided into subclasses on the basis of relatively minor differences in CH sequence and function, e.g., humans express the following subclasses: IgG1, IgG2, IgG3, IgG4, IgA1, and IgA2.

The term “variable” refers to the fact that certain segments of the variable domains differ extensively in sequence among antibodies. The V domain mediates antigen binding and define specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen binding. The hypervariable region generally comprises amino acid residues from a “complementarity determining region” or “CDR” (e.g. around about residues 24-34 (L1), 50-56 (L2) and 89-97 (L3) in the V L, and around about 1-35 (H1), 50-65 (H2) and 95-102 (H3) in the VH; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the V L, and 26-32 (H1), 53-55 (H2) and 96-101 (H3) in the VH; Chothia and Lesk J. Mol. Biol. 196:901-917 (1987)).

Polyclonal antibodies are preferably raised in animals by multiple subcutaneous (sc) or intraperitoneal (ip) injections of the relevant antigen and an adjuvant. It may be useful to conjugate the relevant antigen (especially when synthetic peptides are used) to a protein that is immunogenic in the species to be immunized. For example, the antigen can be conjugated to keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl2, or R1N═C═NR, where R and R1 are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later, the animals are boosted with □ to 1/10 the original amount of peptide or conjugate in Freund's complete adjuvant by subcutaneous injection at multiple sites. Seven to 14 days later, the animals are bled and the serum is assayed for antibody titer. Animals are boosted until the titer plateaus. Conjugates also can be made in recombinant cell culture as protein fusions. Also, aggregating agents such as alum are suitably used to enhance the immune response.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495-(1975. Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal e.g, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs. 130:151-188 (1992), see also U.S. Pat. No. 4,816,567. The “monoclonal antibodies” including antibody fragments may also be isolated from phage antibody libraries using the techniques described in McCafferty et al., Nature, 348:552-554 (1990) Clackson et al., Nature, 352:624-628 (1991) and Marks et al., J. Mol. Biol., 222:581-597 (1991), for example. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res. 21:2265-2266 (1993)). Thus, these techniques are viable alternatives to traditional monoclonal antibody hybridoma techniques for isolation of monoclonal antibodies

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc), and human constant region sequences.

An “intact” antibody is one which comprises an antigen-binding site as well as a CL and at least heavy chain constant domains, CH1, CH2 and CH3. The constant domains may be native sequence constant domains (e.g. human native sequence constant domains) or amino acid sequence variant thereof. Preferably, the intact antibody has one or more effector functions.

“Antibody fragments” comprise a portion of an antibody, preferably the antigen binding or variable region of the intact antibody. Examples of antibody fragments include Fab, Fab′, F(ab′)2, and Fv fragments; diabodies; linear antibodies (see U.S. Pat. No. 5,641,870, Example 2; Zapata et al., Protein Eng. 8(10): 1057-1062 [1995]); single-chain antibody molecules; and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having divalent antigen-binding activity and is still capable of cross-linking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain, such as a Camelid VHH domains that occur naturally in Camelids (or half of an Fv comprising only three CDRs specific for an antigen) has the ability to recognize and bind antigen, although this may be at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

The antibodies of the invention may further comprise “humanized” antibodies or human antibodies. Humanized forms of non-human (e.g., murine) antibodies are chimeric immunoglobulins, immunoglobulin chains or fragments thereof (such as Fv, Fab, Fab′, F(ab′) 2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances; Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity and HAMA response (human anti-mouse antibody) when the antibody is intended for human therapeutic use. According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (Sims et al., J. Immunol. 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol. 151:2623 (1993)). It is further important that antibodies be humanized with retention of high binding affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Various forms of a humanized anti-FZD8 polypeptide antibody are contemplated. For example, the humanized antibody may be an antibody fragment, such as a Fab, which is optionally conjugated with one or more cytotoxic agent(s) in order to generate an immunoconjugate. Alternatively, the humanized antibody may be an intact antibody, such as an intact IgG1 antibody.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J H) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immuno. 7:33 (1993); U.S. Pat. Nos. 5,545,806, 5,569,825, 5,591,669 (all of GenPharm); U.S. Pat. No. 5,545,807; and WO 97/17852.

Preferably, phage display technology (McCafferty et al., Nature 348:552-553 [1990]) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

As described herein, antibodies that are antagonist antibodies of Fzd8 or its Wnt ligands, e.g. Wnt8a are particularly useful in the practice of the invention. Fzd8 antagonist antibodies (against human Fzd8) will be particularly useful in the treatment of osteoarthritis, preferably these are human or humanized monoclonal antibodies. Suitable procedures for generating antibodies are well know to those skilled in the art and a number of these methods are referred to herein. Preferably, the CRD (cysteine rich domain) domain of human Fzd8 (as described herein) which comprises the N-terminal 1-120 amino acids is used to generate Fzd8 antagonist antibodies. The CRD domain can be produced, for example, in a manner analogous to that described in Dann et al Nature 412 (2001) 86-90. Preferably, for therapeutic applications, phage display is used to generate human Fzd8 antagonist antibodies.

### Pharmaceutical Compositions

Another aspect of the invention relates to a pharmaceutical composition comprising a Fzd8 or Wnt modulator or candidate compound of the invention and a pharmaceutically acceptable carrier, diluent, excipient or adjuvant or any combination thereof. Even though Fzd8 or Wnt modulators or candidate compounds (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine. In one embodiment the invention relates to a pharmaceutical composition for treating osteoarthritis comprising an effective amount of a Fzd8 antagonist antibody and a pharmaceutically acceptable vehicle, carrier or excipient.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

### Salts/Esters

The modulators or candidate compounds of the present invention can be present as salts or esters, in particular pharmaceutically acceptable salts or esters.

Pharmaceutically acceptable salts of the modulators or candidate compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

### Therapeutic Use

Modulators of Fzd8 or a Wnt polypeptide including those identified in accordance with the methods of the invention have activity as pharmaceuticals, and may be used in the treatment (therapeutic or prophylactic) of conditions/diseases in human and non-human animals which are exacerbated or caused by excessive or unregulated activation of Fzd8. Suitably said diseases include those diseases characterised by beta catenin signalling. In addition, said diseases may include diseases characterised by tissue calcification or mineralisation including, for example, athlerosclerosis.

Examples of such conditions/diseases include OA and other disease of the bone and joints such as rheumatoid arthritis, seronegative spondyloarthropathies (including ankylosing spondylitis, psoriatic arthritis and Reiter's disease), Behcet's disease, Siogren's syndrome and systemic sclerosis, gout and osteoporosis.

Other diseases include cancer and related disorders. In particular, such modulators could be used as antiangiogenic treatment.

Thus, compounds identified in accordance with the invention, or pharmaceutically-acceptable salts or solvates thereof, are for use in therapy.

In the context of the present specification, the term therapy” also includes “prophylaxis” unless there are specific indications to the contrary. The terms “therapeutic” and “therapeutically” should be construed accordingly.

A further aspect of the invention therefore relates to a method of treating a Fzd8-related disorder, particularly osteoarthritis, said method comprising administering to a subject in need thereof a compound identified in accordance with the invention. Preferably the compound is a Fzd8 antagonist antibody, for example, an antibody that binds to the Fzd8 CRD. In one embodiment the invention comprises a method of treating osteoarthritis comprising administering to a human or animal patient an effective amount of a Fzd8 antagonist antibody.

A further aspect of the invention relates to the use of a Fzd8 or Wnt modulator or candidate compound according to the invention in the preparation of a medicament for treating a Fzd8-related disorder. Preferably the modulator is a Fzd8 antagonist antibody, more preferably an antibody that binds to the cysteine rich domain of Fzd8. In another embodiment the modulator comprises the cysteine rich domain of Fzd8. Preferably the Fzd8-related disorder is osteoarthritis (OA).

A further aspect of the invention relates to the use of a composition comprising the cysteine rich domain of human Fzd8 in the preparation of a medicament for use in the treatment of osteoarthritis (OA). In another embodiment the composition comprises a Fzd8 antagonist antibody. In another embodiment the composition comprises a Fzd8 antagonist antibody that binds to the cysteine rich domain of Fzd8.

In another aspect the invention relates to a method of treating a mammal having osteoarthritis (OA), wherein the method comprises administering to the mammal a therapeutically effective amount of a Fzd8 antagonist, thereby resulting in the effective therapeutic treatment of OA. Preferably the Fzd8 antagonist is an antibody, more preferably an antibody that binds to the cysteine rich domain of Fzd8.

As used herein the phrase “preparation of a medicament” includes the use of the compound directly as the medicament in addition to its use in a screening programme for further therapeutic agents or in any stage of the manufacture of such a medicament.

An used herein the phrase “effective amount” of a Wnt or Fzd8 antagonist is an amount sufficient to carry out a specifically stated purpose. An “effective amount” may be determined empirically and in a routine manner, in relation to the stated purpose.

The term “therapeutically effective amount” refers to an amount of a Wnt or Fzd8 antagonist effective to treat a disease or disorder in a subject. In the case of osteoarthritis, the therapeutically effective amount of the antagonist may reduce or halt the progression of osteoarthritis; and/or relieve to some extent one or more of the symptoms associated with osteoarthritis.

Yet another aspect relates to a method of selectively inhibiting a Fzd8 in a cell comprising contacting said cell with an amount of a compound identified in accordance with the invention, such that a Fzd8 is selectively inhibited in said cell. Preferably the compound is a Fzd8 antagonist antibody, more preferably an antibody that binds to the cysteine rich domain of Fzd8. Preferably the cell is a chondrocyte cell.

### Administration

The pharmaceutical compositions of the present invention may be adapted for oral, intraarticular, rectal, vaginal, parenteral, intramuscular, intraperitoneal, intraarterial, intrathecal, intrabronchial, subcutaneous, intradermal, intravenous, nasal, buccal or sublingual routes of administration.

For ease of administration, it is preferable to be able to deliver the compositions systemically. For an antagonist antibody this could, for example, be via the subcutaneous or intravenous route. However, it may, in the treatment of osteoarthritis, be preferable to deliver the antagonist antibody locally to the site of disease, e.g. intraarticular administration.

For oral administration, particular use is made of compressed tablets, pills, tablets, gellules, drops, and capsules. Preferably, these compositions contain from 1 to 250 mg and more preferably from 10-100 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, to pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose.

### Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.01 to 30 mg/kg body weight, such as from 0.1 to 10 mg/kg, more preferably from 0.1 to 1 mg/kg body weight.

In an exemplary embodiment, one or more doses of 10 to 150 mg/day will be administered to the patient for the treatment of malignancy.

### Methods of Diagnosis by Detecting Fzd8 Expression

Diagnostic assays can measure the amount of Fzd8 and/or Wnt polypeptide, genes or mRNA in a sample of tissue. Assays that measure the amount of mRNA encoding either or both of these polypeptides also fit in this category. Assays can also evaluate the qualities of the receptor or the ligand. For example, assays that determine whether an individual expresses a mutant or variant form of either Fzd8 and/or Wnt or both, can be used diagnostically. In addition, assays that measure one or more activities of Fzd8 and/or Wnt polypeptide can be used diagnostically.

### Kits

The invention provides for kits useful for screening for modulators of Fzd8 or Wnt activity, as well as kits useful for diagnosis of diseases or disorders characterized by dysregulation of Fzd8 or Wnt signaling. Kits useful according to the invention can include an isolated Fzd8 polypeptide (including a membrane- or cell-associated Fzd8 polypeptide, e.g., on isolated membranes, cells expressing Fzd8, or, on an SPR chip) and an isolated Wnt polypeptide.

A kit can also comprise an antibody specific for Fzd8, particularly an antagonist of Fzd8, and/or an antibody for a Wnt polypeptide.

Alternatively, or in addition, a kit can contain cells transformed to express a Fzd8 polypeptide and/or cells transformed to express a Wnt polypeptide. In a further embodiment, a kit according to the invention can contain a polynucleotide encoding a Fzd8 polypeptide and/or a polynucleotide encoding a Wnt polypeptide. In a still further embodiment, a kit according to the invention may comprise the specific primers useful for amplification of Fzd8 or a Wnt polypeptide as described below. All kits according to the invention will comprise the stated items or combinations of items and packaging materials therefor. Kits will also include instructions for use.

The invention will now be further described by way of Examples, which are meant to serve to assist one of ordinary skill in the art in carrying out the invention and are not intended in any way to limit the scope of the invention.

### EXAMPLES

### Example 1

### Materials and Methods

### Plasmids

The full-length cDNA sequences of Wnt8a and 8b (EMBL Acc No AB057725 and AB073637 respectively) were cloned by RT-PCR from foetal brain RNA. The full-length human Fzd8 sequence (EMBL Acc No AB043703) was cloned on a PCR fragment from BAC clone RP11-425A6 (Invitrogen) (EMBL Acc No HSBA425A6). Full-length sFRP3 cDNA (EMBL Acc No U24163) was cloned by RT-PCR from cartilage RNA. In all cases the cDNAs were cloned by introduction of cloning sites via PCR to allow insertion into the multicloning site of vector pcDNA3.1 (Invitrogen)

### Antibodies and Recombinant Protein

Polyclonal affinity purified anti-Fzd8 antibodies-were raised in rabbits immunised with a 19-mer peptide derived from human Fzd8 primary amino acid sequence. Goat anti-human/mouse/rat β-catenin affinity-purified polyclonal Ab was obtained from R&D systems. Goat anti-human/mouse/rat vimentin polyclonal IgG was from Santa Cruz. Mouse monoclonal anti-goat/sheep IgG-Peroxidase and goat anti-rabbit IgG (whole molecule)-peroxidase antibody were purchased from Sigma. Finally, Alexa Fluor® 488 rabbit anti-goat IgG was from Molecular Probes.

### Cell Culture

CHOK1-Fzd8 cell lines were generated by stable transfection of Fzd8-pcDNA3.1 and were maintained in Dulbecco's Modified Eagle's Media (Sigma) supplemented with 10% FCS, 2 mM L-glutamine, 200U Penicillin (200 μg/ml), streptomycin (1 mg/ml) and geneticin (1 mg/ml) [DMEM culture media]. Primary chondrocytes were isolated from cartilage of osteoarthritic patients undergoing total knee replacement. Cartilage was digested in collagenase (2 mg/ml) in a humidified incubator maintained at 37° C. for 24 h. Cells were washed twice then passed through a sterile cell strainer (100 μm). Recovered cells were resuspended DMEM culture media.

### Transfections

siRNA duplexes (SMART duplexes) directed against Fzd8 and GAPDH were supplied by Dharmacon and transfected into primary chondrocytes using Atufect lipid (Atugen). Knockdown of Fzd8 mRNA was measured at 72 h post-transfection by quantitative RT-PCR.

### RNA Isolation

Osteoarthritic cartilage was obtained from consented total knee joint replacement samples or consented knee articular cartilage taken post mortem. Cartilage was snap frozen and ground under liquid nitrogen using a Glen Creston Spex mill. RNA was extracted from the ground cartilage using a standard TRIzol extraction method (Invitrogen) following manufacturer's protocols. The RNA was purified using a Qiagen RNeasy minicolumn (Qiagen), and treated with DNase. RNA was quantified using an Agilent Bioanalyser 2100 with the RNA Nano6000 chip.

### Quantitative PCR

TaqMan real-time quantitative polymerase chain reaction (PCR) assay was performed on an ABI Prism 7700 Sequence Detection System, according to the manufacturer's protocol (Applied Biosystems). TaqMan RT-PCR assay primers and probes employed are listed in Table 1.

TABLE 1

| | | | |
|---|---|---|---|
| Gene | Forward Primer | Reverse Primer | Probe (5′FAM, 3′Methyl Red) |

| | | | |
|---|---|---|---|
| AGC1 | TGGAAACGTGAATCAGA | GGTAACAGTGGCCCTG | CCCTCCTCACATACCTCCTGGTG |
| | ATCAACT | GTACTT | TGCA |
| LECT1 | GAACTCTGCGGTGACCT | TCTCTTCTTTCCCTCTG | TTTCTGGCTTAAACCAACCTATC |
| | TCCT | GATTTCTT | CA |
| COL2A1 | GCCAACGTCCAGATGAC | TCTTGCAGTGGTAGGTG | CTACGCCTGCTGTCCACGGAA |
| | CTT | ATGTTCT | |
| FBLN1 | TGGATGGCAGGTCATGT | GCCGTAGACGTTGGCA | TGCAGCAGCAGCCCCTGTAGC |
| | GAA | CACT | |
| SOX9 | TTCCGCGACGTGGACAT | GGTCAAACTCGTTGAC | CAGCGACGTCATCTCCAACATCG |
| | | ATCGAA | AGA |
| Fzd1 | CTGCTGGCCGGCTTTGT | GTGCCATCGTGCTTCAT | TCTTCCGCATCCGCAC |
| | | GAT | |
| Fzd5 | CGAGCACAACCACATCC | AGAAGTAGACCAGGAG | CCCTGCACTGTGCAC |
| | ACTA | GAAGACGAT | |

25 ng of RNA was mixed with relevant forward/reverse primer, fluorescent probes and Taqman Quantitect Probe Master-Mix and RT enzyme mix (Qiagen). Samples were incubated for an initial reverse transcription reaction at 50° C. for 30 minutes and then at 95° C. for 15 minutes, followed by 40 cycles at 95° C. for 15 seconds and 60° C. for 1 minute. Relative quantitation of target RNA was carried out by Taqman using SDS v1.9 software.

Taqman RT-PCR using Applied Biosystems Low Density Arrays (Taqman Microfluidics) was performed on the ABI Prism 7900. Transcriptional effects following infection of primary chondrocytes with Adv-Wnt8a were measured against a panel of primer/probe sets (Applied Biosystems Assays on Demand). Each test RNA sample was added to Quantitect probe RT-PCR mastermix (Qiagen) at a concentration of 1 ng/μl and applied to the card according to the manufacturer's instructions. One step RT-PCR was carried out with an initial reverse transcription reaction at 50° C. for 30 minutes, 94.5° C. for 15 minutes, followed by 35 cycles at 97° C. for 30 seconds and 59.7° C. for 1 minute. Data analysis was carried out using the Applied Biosystems SDS 2.1 software and transcriptional changes represented as fold change (2^{-ΔΔCt}) with respect to the control virus infected sample.

### Western Blots

Total protein was isolated from cell lines or primary chondrocytes using RIPA buffer (0.15 M NaCl, 0.05 M Tris-HCl pH 7.4, 1 mM EDTA, 1% v/v Triton X100, 0.1% w/v SDS, 1% deoxycholate) containing 1:100 dilution of protease inhibitor cocktail (Sigma). Samples were diluted in SDS sample buffer with β-Mercaptoethanol and 151 g protein loaded onto a 4-12% Bis-Tris gels in MES SDS running buffer and run at 200v for 30 min. Samples were transferred onto PVDF using a semi-dry system. Membranes were blocked for 1 hour in 5% marvel/PBS. Anti-Fzd8 primary antibody was applied at 1:100, β-catenin primary antibody was applied at 1:3000 and vimentin primary antibody at 1:500. Secondary antibodies were used at 1:10,000 in 1% marvel/PBS. Blots were developed using Amersham ECL reagent according to manufacturer's protocols.

### Adenoviral (Adv) Infections

Wnt8a adenovirus (Adv-Wnt8a) and a control virus expressing red fluorescent protein (Adv-RFP) were used for gene delivery. Replication-defective adenovirus (Ad5 c20) encoding full length Wnt8a and RFP (control) were produced by Galapagos Genomics (2301 CA Leiden, The Netherlands). Primary chondrocytes at passage 2 following isolation were transduced with adenovirus at a multiplicity of infection of 200:1 and 500:1 for 6 hours. Cells were harvested for RNA isolation at between 24 h to 8 weeks post-infection.

### Immunohistochemistry

5 mm cartilage biopsies were obtained from human OA and PM donors and snap frozen in OCT Embedding Medium (Raymond Lamb) and stored at −80° C. 7 μm sections were cut and mounted onto Superfrost+ slides (VWR). Sections were fixed in cold acetone (10 min), washed (TBS 0.05% Tween 20) and hyaluronidase treated (0.5 mg/ml Hyaluronidase, Sigma in PBS, 20 min, room temperature). Sections were washed and peroxidase blocked in 3% H₂O₂ (Aldrich) in methanol (30 min). Sections were washed and protein blocked in 20% sheep serum (Dako in TBS 0.05% Tween 20 1% BSA, 60 min, room temp.). Rabbit anti-Fzd 8 (1:50) was pre-incubated either alone or with Fzd-8 peptide (0.65 mg/ml in PBS, 1:50, 30 min) before adding to the sections (overnight, 4° C.). Sections were washed (TBS 0.05% Tween 20) then incubated with biotinylated sheep anti-rabbit antibody (Serotec, 1:200, 30 min, room temp.). Sections were washed (TBS 0.05% Tween 20) and incubated with Streptavidin ABC-HRP diluted according to the manufacturers instructions (Dako, 30 min, room temp.), washed (TBS 0.05% Tween 20) and developed using DAB chromogen (Dako, 5 min). Sections were counterstained using Gills II Haematoxylin (Pioneer Research Chemicals, PRC/13/1), dehydrated to xylene and mounted.

### Immunofluorescence (Arrayscan)

Cells were fixed in 3.7% formaldehyde and permeabilised in PBS/0.05% Triton X100. β-catenin primary antibody was applied at 1:100 and the fluorescent conjugated secondary antibody at 1:400 with Hoechst nuclear stain at 0.5 μg/ml. Cells were visualised using the Arrayscan HCS system (Cellomics) and nuclear staining quantitated using the manufacture's software.

### Results

### Expression of Fzd8 is Mainly Restricted to Cartilage and is Upregulated in Osteoarthritic Cartilage

Quantitative RT-PCR was performed to establish expression of Fzd8 in a range of human tissues. Fzd8 was found to have a restricted expression profile with high levels in cartilage and low or negligible levels in other tissues of the body [FIG. 1]. Analysis of expression levels of Fzd8 mRNA in a panel of 9 osteoarthritic cartilage samples (taken from patients undergoing total knee replacement) and normal cartilage taken post mortem revealed enhanced expression of Fzd8 in osteoarthritic cartilage [FIG. 2].

Localisation of Fzd8 receptor expression in osteoarthritic and normal cartilage was established using immunohistochemistry and Fzd8 was found to be expressed by chondrocytes throughout the full thickness of the cartilage with higher levels of expression in the mid to deep zone in several samples [FIG. 3].

Wnt8a is expressed in normal and osteoarthritic cartilage

Quantitative RT-PCR was carried out to establish expression of Wnt8a in a range of human tissues including tissues from the joints of osteoarthritic patients and normal joints taken post mortem. Highest expression of Wnt8a was found in testis with expression also seen in brain and normal and osteoarthritic cartilage with lower levels of expression in bone, synovium and meniscal cartilage [FIG. 4].

Human Wnt8a and Human Wnt8b are Able to Mediate β-Catenin Accumulation and Nuclear Translocation, but Only when Cotransfected into Cell Lines with Human Fzd8.

In order to establish whether Fzd8 is the receptor for Wnt8a or Wnt8b we performed cotransfections of Wnt8 and Fzd8 in CHOK1 cell lines and measured β-catenin accumulation.

CHOK1 cell lines or CHOK1 cells stably expressing human Fzd8 were transiently transfected with human Wnt8a or Wnt8b constructs. Cells were lysed at 48 hours post transfection and β-catenin measured by Western Blot [FIG. 5]. There was no effect of Wnt8a on β-catenin accumulation in CHOK1 cells however, β-catenin did accumulate when Wnt8a was transfected into Fzd8:CHOK1 cells. β-catenin levels were not modified by control RFP in either cell type. In addition, nuclear localisation of β-catenin (as measured by β-catenin immunofluorescence) was induced in Fzd8:CHOK1 cells infected by an adenovirus expressing Wnt8a but not by a control virus [FIG. 6].

Wnt8a Dependent β-Catenin Accumulation is Blocked by Overexpression of Human sFRP3 (FrzB).

sFRP3 (FrzB) has homology with the cysteine rich domain of Fzd8 and is an endogenous antagonist of Wnt signalling. Cotransfections of Wnt8a and sFRP3 into the Fzd8:CHOK1 cell line were monitored for β-catenin accumulation. The Wnt8a mediated β-catenin accumulation in Fzd8:CHOK1 cells was blocked by overexpression of sFRP3 [FIG. 7].

### Exogenous Expression of Human Wnt8a in Human OA Chondrocytes Leads to Increased β-Catenin Accumulation and Modulation of Gene Expression.

OA articular chondrocytes were transfected with adenovirus encoding Wnt8A (Adv-Wnt8a) or control (Adv-RFP) adenovirus. β-catenin was found to accumulate (as measured by western Blot) 72 h post-infection. Control virus had no affect on β-catenin accumulation [FIG. 8].

β-catenin is known to mediate gene expression through the TCF/LEF family of transcriptional activators/repressors. In order to study the effects of Wnt8a signalling through β-catenin in osteoarthritic chondrocytes, Wnt8a was overexpressed by Adv-Wnt8a infection and a panel of gene expression changes were monitored by quantitative RT-PCR. Altered gene expression was studied over a period of 8 weeks and was indicative of an altered chondrocyte phenotype. Wnt8a suppressed chondrocyte genes [Col α1(II), Col α1(IX), Col α1(XI), SOX9, aggrecan] and induced the expression of genes associated with chondrocyte hypertrophy and calcification [Col α1(X), fibulin and bone matrix GLA protein]. In addition, the antiangiogenic gene chondromodulin was downregulated while the pro-angiogenic gene VEGF was upregulated by Wnt8a overexpression [Table 2].

TABLE 2

| | Fold- | Fold- |
|---|---|---|
| Gene | Induction | Reduction |
| Collagen alpha I (II) | | 25.0 |
| Collagen alpha 1 (IX) | | 43.5 |
| Collagen alpha 1 (XI) | | 5.3 |
| SOX9 | | 4.1 |
| Aggrecan | | 20.0 |
| Chondromodulin | | 9.6 |
| Collagen alpha 1 (X) | 2.2 | |
| Bone matrix GLA Protein | 1.4 | |
| Fibulin | 6.4 | |
| VEGF | 5.2 | |

siRNA Targeted Against Fzd8 mRNA is Able to Block the Effects of Wnt8a Mediated Gene Expression in Human OA Chondrocytes

Primary chondrocytes were stimulated with Wnt8a (Adv-Wnt8a infection) following transfection with Fzd8 siRNA. siRNA versus Fzd8 knocked down Fzd8 mRNA expression by 75-85% as determined by quantitative PCR, but had minimal effect on the knockdown of other Fzd receptors expressed in cartilage chondrocytes, namely Fzd1, Fzd5 and Fzd7. Data reveal that Fzd8 siRNA prevented Wnt8a regulated gene changes [e.g. Fzd8 siRNA blocked Wnt8a mediated SOX9 reduction by 52%, FIG. 9].

These data are consistent with a role for Wnt8a in regulating cartilage chondrocyte phenotype in osteoarthritis. Based on our broad expression analysis, Fzd8 has a relatively restricted expression profile, however it is highly expressed in articular cartilage and expression is increased further in osteoarthritic cartilage. Fzd8 is a receptor for Wnt8a, as demonstrated by recombinant cell line and primary chondrocyte experiments, and induces the stabilisation of β-catenin to regulate gene expression in the nucleus. Primary articular cartilage chondrocytes stimulated with Wnt8a respond by modulating the expression of genes associated with a change in phenotype from a resting state to a more hypertrophic state, a change normally associated with the process of endochondral ossification.

Endochondral ossification is the formation of calcified bone following remodelling of a cartilage scaffold model and is the normal process of long bone growth during development. Cartilage chondroyte phenotype changes occur during this process and lead to the accumulation of mature hypertrophic chondrocytes that express and secrete matrix proteins such as collagen X, resulting in a mineralised extracellular matrix and ultimately the formation of new bone. In normal, fully developed articular cartilage, where further endochondral ossification is undesirable, chondrocyte differentiation is negatively regulated. However, in osteoarthritis, this regulation is apparently lost and deep zone calcified cartilage, found at the interface between non-mineralised articular cartilage and bone, expands and extends into the deep zone cartilage, compromising the mechanical properties of the overlying cartilage matrix. Thus our data suggest a role for the Wnt8/Fzd8 pathway in stimulating articular cartilage chondrocyte hypertrophy leading to a more calcified deep zone extracellular matrix that ultimately contributes to the loss of cartilage integrity, cartilage erosion and the joint space narrowing observed during osteoarthritis.

### Example 2

### Materials and Methods

### TCF/LEF Reporter Assay

Cells stably expressing Fzd8 were transfected with a firefly luciferase TCF reporter plasmid [Topflash] and a constitutively expressed renilla luciferase, as a normalization control. Control cells were transfected with a reporter plasmid in which the TCF binding sites have been mutated [Fopflash] as a negative control (reporter plasmids available from upstate). Cells were stimulated with 10-50NN Wnt3a protein (R&D systems) 24 h after transfection. Luciferase activity was determined 24 h after treatment with Wnt3A. The cells were lysed and assayed for firefuly and renilla luciferase activities using the dual luciferase assay kit, following the manufacturers protocol (Promega corp).

### Results

### Screening for Fzd8 Activity Using the TCF/LEF Reporter Assay

In order to establish a method for screening Fzd8 we treated cells stably expressing Fzd8 and a TCF reporter plasmid with recombinant Wnt3a protein and measured luciferase activity.

CHOK1 cells and CHOK1 cells stably expressing human Fzd8 were transiently transfected with a firefly luciferase TCF reporter plasmid and a constitutively expressed Renilla luciferase, as a normalization control. Cells were treated with Wnt3a after 24 hours and luciferase activity measured after a further 24 hours. There was little effect of Wnt3a on luciferase activity in CHOK1 cells however, luciferase activity was increased 10.4 fold when 20 ng Wnt3a was applied to Fzd8:CHOK1 cells. Luciferase activity was not increased in either cell type transfected with a reporter plasmid where the TCF binding sites have been mutated. All results are normalized to the Renilla luciferase activity. [FIG. 10].

### Example 3

There are Fzd8 CRD antibodies commercially available (R&D Systems) but these are suitable for detection only and have no neutralising (blocking or antagonistic) activity.

Therefore, in order demonstrate that an antibody approach to block (antagonise) Wnt/Fzd8 signalling, e.g by using a Fzd8 antagonist antibody, it was demonstrated that the Fzd8 CRD domain is important in ligand binding and activation of the receptor.

In this experiment we have used the Fzd8 CRD to block binding and signalling of Wnt3a through the Fzd8 receptor and looked at effects on a for β-catenin reporter. A for β-catenin reporter was used as a surrogate for β-catenin accumulation, which correlates with OA pathology. This experiment shows that the Fzd8 CRD does indeed block Wnt3a mediated effects and demonstrated that it would be feasible to screen and identify a Fzd8 antagonist (blocking) antibody by raising an antibody against the soluble Fzd8 CRD domain.

### Materials and Methods.

Polyclonal affinity purified anti-Fzd8 antibodies were raised in rabbits immunised with a 19-mer peptide derived from human Fzd8 primary amino acid sequence. Goat anti-human/mouse/rat β-catenin affinity-purified polyclonal Ab was obtained from R&D systems. Goat anti-human/mouse/rat vimentin polyclonal IgG was from Santa Cruz. Mouse monoclonal anti-goat/sheep IgG-Peroxidase and goat anti-rabbit IgG (whole molecule)-peroxidase antibody were purchased from Sigma. Recombinant Wnt3a and murine Fzd8 CRD Fc fusion was obtained from R&D systems. Finally, Alexa Fluor® 488 rabbit anti-goat IgG was from Molecular Probes.

### Results

### The Fzd8 CRD Affects Wnt3a Mediated Activation of the Fzd8/Beta-Catenin Response

We treated cells stably expressing Fzd8 and a TCF reporter plasmid with recombinant Wnt3a protein and increasing concentrations of the Fzd8 CRD Fc fusion and measured luciferase activity. Cells were treated with Wnt3a preadsorbed with the Fzd CRD and luciferase activity measured after 24 hours. The increase in luciferase activity in response to Wnt3a can be inhibited by the R&D soluble mFzd8 CRD with an ND50 of 0.027 μg/ml [FIG. 11].

1-4. (canceled)

5. A method of treating a mammal having osteoarthritis (OA), wherein the method comprises administering to the mammal a therapeutically effective amount of a Fzd8 antagonist, wherein the Fzd8 antagonist inhibits the ability of Fzd8 to bind Wnt8a.

6. A method according to claim 5 wherein the Fzd8 antagonist is an antibody.

7. A method according to claim 6 wherein the antibody binds to the cysteine rich domain of Fzd8.

8. A method of preparing a pharmaceutical composition comprising an agent that modulates the function of Fzd8 for use in the treatment of OA, said method comprising:
a) contacting a Fzd8 polypeptide with a Wnt8a polypeptide in the presence or absence of a candidate modulator under conditions permitting the binding of said Wnt8a polypeptide to said Fzd8 polypeptide; and
b) measuring the binding of said Fzd8 polypeptide to said Wnt8a polypeptide, wherein a decrease in binding in the presence of said candidate modulator, relative to the binding in the absence of said candidate modulator, identifies said candidate modulator as an agent that modulates the function of Fzd8; and
c) preparing a pharmaceutical composition comprising said agent that modulates the function of Fzd8 and a pharmaceutically acceptable carrier, diluent, excipient or adjuvant or any combination thereof.

9. A method of preparing a pharmaceutical composition comprising an agent that modulates the function of Fzd8 for use in the treatment of OA, said method comprising:
a) contacting a Fzd8 polypeptide with a Wnt8a polypeptide in the presence or absence of a candidate modulator under conditions permitting the binding of said Wnt8a polypeptide to said Fzd8 polypeptide; and
b) measuring the signalling activity of said Fzd8 polypeptide to said Wnt8a polypeptide, wherein a change in activity in the presence of said candidate modulator, relative to the activity in the absence of said candidate modulator, identifies said candidate modulator as an agent that modulates the function of Fzd8; and
c) preparing a pharmaceutical composition comprising said agent that modulates the function of Fzd8 and a pharmaceutically acceptable carrier, diluent, excipient or adjuvant or any combination thereof.

10. A method as claimed in claim 9 wherein Fzd8 signalling activity is measured by measuring the accumulation, nuclear translocation or phosphorylation status of β-catenin.

11. A method as claimed in claim 8 wherein said method is a cellular assay.

12. A method as claimed in claim 11 wherein said cellular assay comprises co-transfecting a cell or cell line with Fzd8 and Wnt8a.
